(19) United States
(12) Patent Application Publication
(10) Pub. No.: US 2021/0270780 A1
(43) Pub. Date: Sep. 2, 2021

(54) PHOTOACOUSTIC DUAL-MODE IMAGING PROBE

(71) Applicants: SHENZHEN MINDRAY BIO-MEDICAL ELECTRONICS CO., LTD., Shenzhen (CN); SHENZHEN MINDRAY SCIENTIFIC CO., LTD., Shenzhen (CN)

(72) Inventors: Fei WU, SHENZHEN (CN); Ming TANG, SHENZHEN (CN); Changxing KE, SHENZHEN (CN); Fang YANG, SHENZHEN (CN)

(21) Appl. No.: 17/204,185

(22) Filed: Mar. 17, 2021

Related U.S. Application Data
(63) Continuation of application No. PCT/CN2018/106480, filed on Sep. 19, 2018.

(51) Int. Cl. G01N 29/24; A61B 5/00; G01N 21/17; G01H 9/00

(52) U.S. Cl. CPC G01N 29/2418; G01H 9/004; G01N 21/1702; A61B 5/0095

(57) ABSTRACT

Disclosed by the present disclosure is a photoacoustic dual-mode imaging probe, which comprises an optical fiber, a transducer and a housing; the optical fiber and the transducer are at least partially housed at the interior of the housing, and a light outlet of the optical fiber and a front end of the transducer are both located at an head end of the photoacoustic dual-mode imaging probe; the optical fiber is used to emit laser pulses; and the transducer is used to transmit and receive ultrasound signals. The photoacoustic dual-mode imaging probe uses the housing to wrap the optical fiber and the transducer inside the housing, such that the three become a whole, thereby being easy to clean and disinfect, being convenient to hold, having strong human-computer interaction performance, and eliminating the use of a coupling pad.

### TECHNICAL FIELD

The present disclosure relates to a photoacoustic dual-mode imaging probe.

### BACKGROUND

Photoacoustic dual-mode imaging is a dual-mode imaging method that combines photoacoustic imaging and ultrasound imaging. The photoacoustic imaging can represent the functional information of the organism, while the traditional ultrasound imaging can represent the structural information of the organism. They are effectively combined. Therefore, the photoacoustic dual-mode imaging overcomes the shortcomings of single-mode imaging and can provide more comprehensive structural and functional information of the tissue.

The photoacoustic dual-mode imaging system includes an ultrasound device, a laser, and an optical fiber bundle coupled to an ultrasound probe. The photoacoustic system and the ultrasound system are relatively independent and can be separated. It is difficult to clean and disinfect during use, and the grip and human-computer interaction performance are poor. In addition, during the use of the photoacoustic dual-mode imaging system, a coupling pad needs to be used to concentrate the laser energy under the transducer while diffusing the laser spot. The coupling pad needs to be cleaned, disinfected and replaced, which increases the use and maintenance costs.

### SUMMARY

The present disclosure provides a photoacoustic dual-mode imaging probe to address the problems that, when the photoacoustic dual-mode imaging system is used, it is difficult to clean and disinfect and the grip and human-computer interaction performance are poor during use due to the relative independence between the photoacoustic system and the ultrasound system of the probe, and the use of the coupling pad brings many inconveniences.

The present disclosure provides a photoacoustic dual-mode imaging probe, which may include an optical fiber, a transducer, and a housing. The optical fiber and the transducer may be at least partially housed in the housing. The light outlet of the optical fiber and the transducer may be both located at the head end of the photoacoustic dual-mode imaging probe. The optical fiber may be used to transmit the laser. The transducer may be used to transmit and receive the ultrasound signals.

In the photoacoustic dual-mode imaging probe provided by the present disclosure, the optical fiber and the transducer may be housed inside the housing, such that the optical fiber, and transducer and the probe are a whole, which is convenient for cleaning and disinfection, is convenient for holding, has better human-computer interaction performance, and eliminates the need for coupling pad.

### BRIEF DESCRIPTION OF THE DRAWINGS

FIG. 1 is a cross-sectional view of a photoacoustic dual-mode imaging probe in one embodiment;

FIG. 2 is a partial enlarged view of a cross-section of a photoacoustic dual-mode imaging probe in one embodiment; and

FIG. 3 is a cross-sectional view of a photoacoustic dual-mode imaging probe in one embodiment.

### DETAILED DESCRIPTION

The technical solutions in the embodiments of the present disclosure will be clearly and completely described below in conjunction with the drawings. Obviously, the described embodiments are only a part, but not all, of the embodiments of the present disclosure. Based on the embodiments of the present disclosure, all other embodiments obtained by those of ordinary skill in the art without creative work shall fall within the protection scope of the present disclosure. In addition, since known functions and configurations may blur the description with unnecessary details, they will not be described in detail.

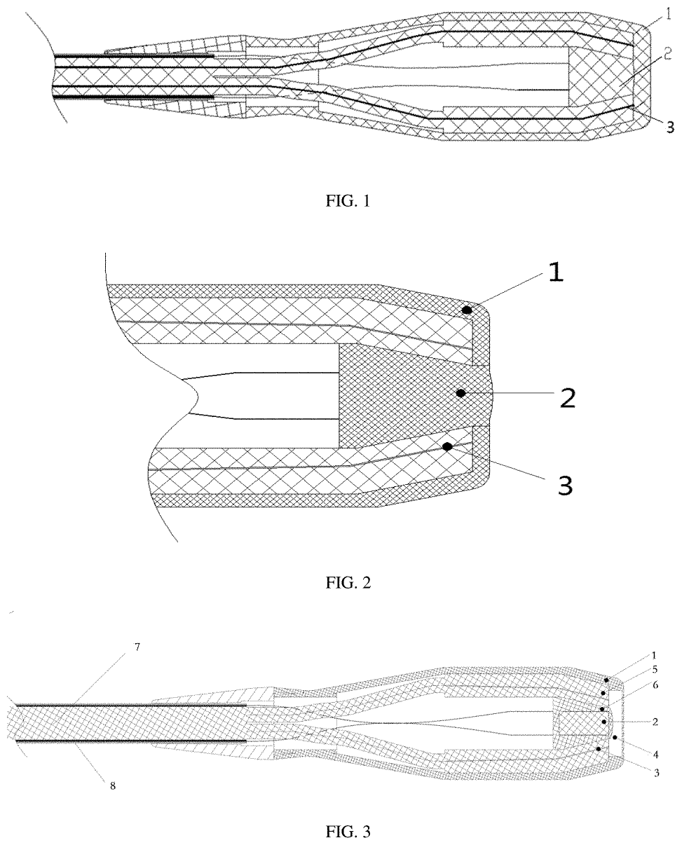

As shown in FIG. 1 and FIG. 2, in one embodiment, the photoacoustic dual-mode imaging probe may include an optical fiber 3, a transducer 2 and a housing 1. The optical fiber 3 may be used to transmit laser pulses. The transducer may be used to transmit and receive ultrasound signals. The optical fiber 3 and the transducer 2 may be at least partially housed in the housing 1. For the convenience of description, the end of the transducer 2 that transmits and receives the ultrasound signals may be referred to as the front end, the end of the photoacoustic dual-mode imaging probe for scanning may be referred to as the head end, and front of the head end may be referred to as front of the photoacoustic dual-mode imaging probe. The light outlet of the optical fiber 3 and the transducer 2 may be both arranged at the head end of the photoacoustic dual-mode imaging probe, so as to realize the functions of transmitting the laser pulses and transmitting and receiving the ultrasound signals of the head end of the photoacoustic dual-mode imaging probe. The optical fiber 3 and the transducer 2 may be at least partially housed in the housing 1. The optical fiber 3 and the transducer 2 may be completely housed in the housing 1. Alternatively, the light outlet of the optical fiber 3 and the front end of the transducer 2 may be exposed outside the housing 1 while the rest parts may be housed in the housing 1, that is, the head end of the photoacoustic dual-mode imaging probe may not be housed by the housing 1. Other ways in which the optical fiber 3 and the transducer 2 are at least partially housed in the housing 1 may also be used. When the photoacoustic dual-mode imaging probe responds to the working signal, on the one hand, the optical fiber 3 may transmit the laser pulses to irradiate human tissues, the materials with strong optical absorption properties in the tissues absorb the light energy and cause local heating and thermal expansion, thereby generating ultrasound signals which propagate outwards and received by the transducer 2, and the transducer 2 may convert the received ultrasound signal into electrical signals and transmits the electrical signals to the host where the ultrasound signals may be processed to generate a photoacoustic image for medical staff to diagnose; on the other hand, when receiving the working signal, the transducer 2 may transmit ultrasound signals to the human tissue, receives the corresponding ultrasound echo signals, and convert the received ultrasound signals into electrical signals and transmit the electrical signals to the host where the ultrasound signals may be processed to generate an ultrasound image for medical staff to diagnose. In the photoacoustic dual-mode imaging probe, the optical fiber 3 and the transducer 2 are housed in the housing 1 to form one integrated probe. Therefore, the requirements for the probe for dual-mode imaging of photoacoustic imaging and ultrasound imaging can be met by one single photoacoustic dual-mode imaging probe, which improves the grip of the probe and increase the convenience of cleaning and disinfection during use.

As shown in FIG. 1, in one embodiment, the optical fiber 3 and the transducer 2 may be completely housed in the housing 1. At least the part of the housing at the light outlet of the optical fiber 3 may be made of light permeable material, and at least the part of the housing at front end of the transducer 2 may be made of sound permeable materials. For example, the part of the housing at the light outlet of the optical fiber 3 is made of light permeable materials and the part of the housing at the front end of the transducer 2 is made of sound permeable materials while the other parts of the housing 1 are made of other materials. Alternatively, the parts of the housing 1 at the light outlet of the optical fiber 3 and at the front end of the transducer 2 are made of light permeable and sound permeable materials while other parts of the housing 1 are made of other materials. Alternatively, the entire housing 1 may be made of light permeable and sound permeable materials. Other ways that meet the conditions may also be used. When the photoacoustic dual-mode imaging probe is used, the laser pulses transmitted from the optical fiber 3 may permeate the light permeable part of the housing 1 at the light outlet of the optical fiber 3 and illuminate the human tissue in front of the head of the probe, which reduces the energy loss caused by the shielding of the transducer to the light signals. Furthermore, due to the action of the light permeable part of the housing 1 at the light outlet of the optical fiber 3, the spot of the laser transmitted from the optical fiber 3 may be diffused, which can reduce the energy irradiated to the local body tissues and avoid laser burns to the skin. In this embodiment, the light permeable part of the housing 1 at the light outlet of the optical fiber 3 through which the laser pulses transmitted from the optical fiber 3 pass may concentrate the laser pulse at front of the head and diffuse the laser spot, which achieve the function of the coupling pad. Therefore, the inconvenience that the coupling pad must be used in the traditional dual-mode imaging process can be avoided. After the laser pulses transmitted from the optical fiber 3 enter the human tissue, the material with strong optical absorption characteristics in the tissue absorbs the light energy, which causes local heating and thermal expansion and thereby generate ultrasound signals that propagate outward. The generated ultrasound signals pass through the sound permeable part of the housing 1 at the front end of the transducer 2 and are received by the transducer 2. The ultrasound signals may be processed to form a photoacoustic image. When the photoacoustic dual-mode imaging probe is used, the ultrasound signals transmitted by the transducer 2 may enter the human tissue through the sound permeable part of the housing 1 at the front end of the transducer 2, and the echo signals may pass through the sound permeable part of the housing 1 at the front end of the transducer 2 and be received by the transducer 2. The echo signals may be processed to form an ultrasound image.

As shown in FIG. 2, in one embodiment, the optical fiber 3 may be completely housed in the housing 1. The front end of the transducer 2 may be exposed outside the housing 1, while the rest parts are housed in the housing 1. At least the part of the housing 1 at the light outlet of the optical fiber 3 may be made of light permeable material. The entire housing 1 may be made of light permeable material. Alternatively, only the part of the housing 1 at the light outlet of the optical fiber 3 is made of light permeable material while the rest parts of the housing 1 are made of other materials. Other ways may also be used. When the photoacoustic dual-mode imaging probe is used, the laser pulses transmitted from the optical fiber 3 may permeate the light permeable part of the housing 1 at the light outlet of the optical fiber 3 and illuminate the human tissue in front of the head of the probe. Furthermore, due to the action of the light permeable part of the housing 1 at the light outlet of the optical fiber 3, the spot of the laser transmitted from the optical fiber 3 may be diffused, which can reduce the energy irradiated to the local body tissues and avoid laser burns to the skin. In this embodiment, the light permeable part of the housing 1 at the light outlet of the optical fiber 3 through which the laser pulses transmitted from the optical fiber 3 pass may concentrate the laser pulse at front of the head and diffuse the laser spot, which achieve the function of the coupling pad. Therefore, the inconvenience that the coupling pad must be used in the traditional dual-mode imaging process can be avoided. After the laser pulses transmitted from the optical fiber 3 enter the human tissue, the material with strong optical absorption characteristics in the tissue absorbs the light energy, which causes local heating and thermal expansion and thereby generate ultrasound signals that propagate outward. The generated ultrasound signals may be received by the transducer 2. The ultrasound signals may be processed to form a photoacoustic image. When the photoacoustic dual-mode imaging probe is used, the ultrasound signals transmitted by the transducer 2 may enter the human tissue, and the echo signals may be received by the transducer 2. The echo signals may be processed to form an ultrasound image.

In one embodiment not shown, the probe may also include a head cover. The light outlet of the optical fiber 3 and the front end of the transducer 2 may be exposed outside the housing 1, that is, the head end of the photoacoustic dual-mode imaging probe may not be housed by the housing 1. The head cover may be arranged at front of the light outlet of the optical fiber 3 and the transducer 2, cover the head end of the photoacoustic dual-mode imaging probe and be connected to the housing 1. The head cover may be made of light permeable and sound permeable materials. When the photoacoustic dual-mode imaging probe is used, the laser pulses transmitted from the optical fiber 3 may permeate the head cove and illuminate the human tissue in front of the head of the probe, which reduces the energy loss caused by the shielding of the transducer to the light signals. Furthermore, due to the action of the head cover, the spot of the laser transmitted from the optical fiber 3 may be diffused, which can reduce the energy irradiated to the local body tissues and avoid laser burns to the skin. In this embodiment, the head cover through which the laser pulses transmitted from the optical fiber 3 pass may concentrate the laser pulses at front of the head and diffuse the laser spot, which achieve the function of the coupling pad. Therefore, the inconvenience that the coupling pad must be used in the traditional dual-mode imaging process can be avoided. After the laser pulses transmitted from the optical fiber 3 enter the human tissue, the material with strong optical absorption characteristics in the tissue absorbs the light energy, which causes local heating and thermal expansion and thereby generate ultrasound signals that propagate outward. The generated ultrasound signals may pass through the head cover and be received by the transducer 2. The ultrasound signals may be processed to form a photoacoustic image. When the photoacoustic dual-mode imaging probe is used, the ultrasound signals transmitted by the transducer 2 may enter the human tissue through the head cover, and the echo signals may pass through the head cover and be received by the transducer 2. The echo signals may be processed to form an ultrasound image.

As shown in FIG. 3, in one embodiment in which the optical fiber 3 and the transducer 2 are completely housed in the housing 1, there may be a filling layer 4 between the light outlet of the optical fiber 3 and the front end of the transducer 2 and the housing 1. The filling layer 4 may be made of light permeable and sound permeable materials that have good acoustic and optical transmission properties, such as liquid coupling agents, gel materials or a mixture thereof, or the like. When the photoacoustic dual-mode imaging probe is used, on the one hand, the laser pulses transmitted from the optical fiber 3 may permeate the filling layer 4 and the housing 1, which can better concentratedly illuminate the light field energy to front of the head; on the other hand, the filling layer 4 and the housing 1 that the laser pulses transmitted from the optical fiber 3 permeate can better diffuse the spot of the laser and reduce the local energy, while the transmitting and receiving of the ultrasound signals will not be affected.

In another embodiment, the light outlet of the optical fiber 3 and the front end of the transducer 2 may be exposed outside the housing 1 and covered by the head cover, and there may be a filling layer between the light outlet of the optical fiber and the front end of the transducer and the head cover. The filling layer may be made of light permeable and sound permeable materials that have good acoustic and optical transmission properties, such as liquid coupling agents, gel materials or a mixture thereof, or the like. When the photoacoustic dual-mode imaging probe is used, on the one hand, the laser pulses transmitted from the optical fiber 3 may permeate the filling layer 4 and the head cover, which can better concentratedly illuminate the light field energy to front of the head; on the other hand, the filling layer 4 and the head cover that the laser pulses transmitted from the optical fiber 3 permeate can better diffuse the spot of the laser and reduce the local energy, while the transmitting and receiving of the ultrasound signals will not be affected.

In one embodiment not shown in which the light outlet of the optical fiber 3 is housed in the housing 1, there may be a filling layer between the light outlet of the optical fiber 3 and the outer housing 1. The filling layer may be made of light permeable materials. When the photoacoustic dual-mode imaging probe is used, the laser pulses transmitted from the optical fiber 3 may permeate the filling layer 4 and the housing 1, which can better concentratedly illuminate the light field energy to front of the head. Furthermore, the filling layer 4 and the housing that the laser pulses transmitted from the optical fiber 3 permeate can better diffuse the spot of the laser and reduce the local energy, while the transmitting and receiving of the ultrasound signals will not be affected.

In one embodiment not shown in which the light outlet of the optical fiber 3 and the front end of the transducer 2 are covered by the head cover, there may be a filling layer between the light outlet of the optical fiber 3 and the head cover. The filling layer may be made of light permeable materials. When the photoacoustic dual-mode imaging probe is used, the laser pulses transmitted from the optical fiber 3 may permeate the filling layer 4 and the head cover, which can better concentratedly illuminate the light field energy to front of the head. Furthermore, the filling layer 4 and the head cover that the laser pulses transmitted from the optical fiber 3 permeate can better diffuse the spot of the laser and reduce the local energy, while the transmitting and receiving of the ultrasound signals will not be affected.

As shown in FIG. 1 to FIG. 3, in one embodiment, the light outlet of the optical fiber 3 may be separated from the front end of the transducer 2 by a predetermined distance so as to reduce the shielding of the transducer to the laser pulse transmitted from the light outlet of the optical fiber 3 that affects the quality of the photoacoustic imaging. Furthermore, the predetermined distance between the light outlet of the optical fiber 3 and the front end of the transducer 2 can reduce the interference of the laser pulses transmitted from the optical fiber 3 to the transducer. The predetermined distance may be determined comprehensively by the type and size of the probe and the measurement requirements.

As shown in FIG. 1 to FIG. 3, in one embodiment, the front section of the optical fiber 3 may be parallel to or be at an acute angle with the axis where the transducer 2 is located. The axis where the transducer 2 is located may be the line perpendicular to the front surface of the transducer 2, that is, the line perpendicular to the surface of the transducer 2 where the ultrasound signals are transmitted and received. The front section of the optical fiber 3 may be the part of the optical fiber 3 that is located at the front portion of the photoacoustic dual-mode imaging probe. The front section of the optical fiber 3 being parallel to or being at an acute angle with the axis where the transducer 2 is located may mean that the angle between the front section of the optical fiber 3 and the axis where the transducer 2 is located is greater than or equal to 0 degrees and less than 90 degrees. This angle may be determined according to the needs of clinical detection depth. In the embodiment in which the front section of the optical fiber 3 and the axis where the transducer 2 is located is at an acute angle, the obliquely arranged light outlet of the optical fiber 3 can solve the problem that the light will be blocked by the transducer 2, and furthermore, can enable the laser pulses transmitted from the optical fiber 3 to be effectively concentrated below the head of the probe.

There may be one or multiple optical fibers 3. The multiple optical fibers may be arranged side by side to form an optical fiber bundle. In one embodiment, there are three optical fibers 3. In one embodiment, multiple optical fibers 3 may be arranged at both sides of the transducer 2, be arranged at one side of the transducer 2, or be arranged around the transducer 2. The multiple optical fibers around the transducer 2 may be evenly arranged around the transducer 2, or be divided into three or four groups around the transducer 2, or be arranged at other positions that facilitate the transmitting of the laser pulses from the optical fiber 3 and the transmitting and receiving of the ultrasound signals by the transducer 2. Multiple optical fibers 3 may be arranged to form optical fiber bundles to be arranged at the positions, or be separately arranged at the positions.

As shown in FIG. 3, in one embodiment, the probe may further include a fixing device 5. The optical fiber 3 may be contained in the fixing device 5. The fixing device 5 may be at least partially housed in the housing. The fixing device 5 may be used for fixing and protecting the optical fiber 3.

In one embodiment, the optical fiber 3 and the transducer 2 may be fixedly connected through glue bonding, mechanical fixing or other fixed connection methods such that the relative position of the optical fiber 3 with respect to the transducer 2 is fixed.

As shown in FIG. 3, in one embodiment, the probe may further include a sound permeable element 6. The sound permeable element 6 may at least partially wrap the transducer 2 and extend to the front surface of the transducer 2. At least a part of the optical fiber 3 may be fixedly connected to the transducer 2 through the sound permeable element 6, and the light outlet of the optical fiber 3 may be exposed outside the sound permeable element 6. The sound permeable element 6 may focus the ultrasound signals transmitted by the transducer and permeating the sound permeable element in front of the transducer, which can reduce the loss of the ultrasound signals and improve the quality of the imaging. Parts of all of the other parts of the sound permeable element 6 may wrap the transducer 2. The sound permeable element 6 connects the transducer 2 and the optical fiber 3 such that the transducer 2 and the optical fiber 3 are fixedly connected through the sound permeable element 6.

In one embodiment, the sound permeable element 6 may be made of sound permeable and light-reflecting materials, such as the material obtained by adding non-absorbing and high-scattering substances to the traditional lens material. Alternatively, the sound permeable element 6 may be made of sound permeable and light-absorbing materials. The sound permeable element 6 made of sound permeable and light-reflecting material or sound permeable and light-absorbing material completely or partially wrapping the transducer can prevent the laser pulses transmitted from the optical fiber 3 from entering the transducer 2 and causing interference. Furthermore, the sound permeable element at the front surface of the transducer can focus the ultrasound signals transmitted by the transducer 2.

As shown in FIG. 3, in one embodiment, the probe may further include a signal cable 7. The signal cable 7 may include a transducer signal line (not shown in the figure), an optical fiber extension section (not shown in the figure) and a sheath 8. The transducer signal line may be connected to the transducer 2, and the transducer may receive and transmit signals through the transducer signal line. The optical fiber extension section may be the part of the optical fiber 3 that extends to the signal cable, and the laser pulses may be transmitted to the optical fiber 3 through the optical fiber extension section and transmitted outward from the outlet of the optical fiber 3. The transducer signal line and the optical fiber extension section may be housed in the sheath 8 such that the optical fiber extension section and the transducer signal line are housed as a whole. Therefore, the overall structure is simple and the photoacoustic dual-mode imaging probe is convenient to use.

In one embodiment, the signal cable 7 may further include a protection device (not shown in the figure). The protection device may house the optical fiber extension section, and the sheath 8 may house the protection device. The protection device can prevent the optical fiber from being broken when it is bent to a certain extent with the signal cable.

In one embodiment, the signal cable 7 may further include a shielding net (not shown in the figure). The shielding net may house the transducer signal line and the optical fiber extension section, and the sheath 8 may house the shielding net. The shielding net can prevent the electrical signal and the optical signal from being interfered by the external environment when they are transmitted in the transducer signal line and the optical fiber extension section, so as to improve the transmission efficiency.

In another embodiment, the transducer signal lines may be evenly arranged around the optical fiber extension section and the sheath 8 house the transducer signal lines and the optical fiber extension section, so as to form the signal cable 7.

The specific embodiments have been described above. However, the protection scope of the present disclosure will not be limited thereto. Any modification or alternative that a person skilled in the art can easily obtained according to the description of the present disclosure will be in the protection scope of the present disclosure. Therefore, the protection scope of the present disclosure should be determined according to the claims.

1. A photoacoustic dual-mode imaging probe, comprising an optical fiber, a transducer, and a housing; wherein
the optical fiber and the transducer are at least partially housed in the housing, and a light outlet of the optical fiber and the transducer are both arranged at a head end of the photoacoustic dual-mode imaging probe;
the optical fiber is configured to transmit laser pulses; and
the transducer is configured to transmit and receive ultrasound signals.

2. The photoacoustic dual-mode imaging probe of claim 1, wherein:
the optical fiber and the transducer are completely housed in the housing;
at least a part of the housing at the light outlet of the optical fiber is made of a light permeable material; and
at least a part of the housing at a front end of the transducer is made of a sound permeable material.

3. The photoacoustic dual-mode imaging probe of claim 1, wherein:
the optical fiber is completely housed in the housing;
a front end of the transducer is exposed outside the housing while a section of the transducer excluding the front end of the transducer is housed in the housing; and
at least a part of the housing at the light outlet of the optical fiber is made of a light permeable material.

4. The photoacoustic dual-mode imaging probe of claim 1, further comprising a head cover; wherein:
the light outlet of the optical fiber and a front end of the transducer are exposed outside the housing;
the head cover covers the light outlet of the optical fiber and the front end of the transducer, and is connected to the housing; and
the head cover is made of a light permeable and sound permeable material.

5. The photoacoustic dual-mode imaging probe of claim 2, wherein:
a filling layer is disposed between the light outlet of the optical fiber and the front end of the transducer and the housing; and
the filling layer is made of a light permeable and sound permeable material.

6. The photoacoustic dual-mode imaging probe of claim 4, wherein:
a filling layer is disposed between the light outlet of the optical fiber and the front end of the transducer and the head cover; and
the filling layer is made of a light permeable and sound permeable material.

7. The photoacoustic dual-mode imaging probe of claim 2, wherein:
a filling layer is disposed between the light outlet of the optical fiber and the housing; and
the filling layer is made of a light permeable material.

8. The photoacoustic dual-mode imaging probe of claim 4, wherein:
a filling layer is disposed between the light outlet of the optical fiber and the head cover; and
the filling layer is made of a light permeable material.

9. The photoacoustic dual-mode imaging probe of claim 1, wherein the light outlet of the optical fiber is separated from a front end of the transducer by a distance.

10. The photoacoustic dual-mode imaging probe of claim 1, wherein a front section of the optical fiber is parallel to or at an acute angle with an axis where the transducer is located.

11. The photoacoustic dual-mode imaging probe of claim 1, comprising multiple optical fibers.

12. The photoacoustic dual-mode imaging probe of claim 11, wherein the multiple optical fibers are arranged on both sides of the transducer, are arranged on one side of the transducer, or are arranged around the transducer.

13. The photoacoustic dual-mode imaging probe of claim 1, further comprising a fixing device, wherein:
the optical fiber is contained in the fixing device; and
the fixing device is at least partially housed in the housing.

14. The photoacoustic dual-mode imaging probe of claim 1, wherein the optical fiber is fixedly connected to the transducer.

15. The photoacoustic dual-mode imaging probe of claim 1, further comprising a sound permeable element, wherein:
the sound permeable element at least partially wraps the transducer and extends to a front surface of the transducer;
at least a part of the optical fiber is fixedly connected to the transducer through the sound permeable element; and
the outlet of the optical fiber is exposed outside the sound permeable element.

16. The photoacoustic dual-mode imaging probe of claim 15, wherein the sound permeable element is made of a sound permeable and light-reflecting material or a sound permeable and light-absorbing material.

17. The photoacoustic dual-mode imaging probe of claim 1, further comprising a signal cable, wherein:
the signal cable comprises a transducer signal line, an optical fiber extension section and a sheath;
the transducer signal line is connected to the transducer;
the optical fiber extension section is a portion of the optical fiber that extends to the signal cable from the head end; and
the transducer signal line and the optical fiber extension section are housed in the sheath.

18. The photoacoustic dual-mode imaging probe of claim 17, wherein:
the signal cable further comprises a protection device;
the protection device houses the optical fiber extension section; and
the sheath houses the protection device.

19. The photoacoustic dual-mode imaging probe of claim 17, wherein:
the signal cable further comprises a shielding net;
the shielding net wraps the transducer signal line and the optical fiber extension section; and
the sheath houses the shielding net.

20. The photoacoustic dual-mode imaging probe of claim 17, wherein the transducer signal lines are evenly arranged around the optical fiber extension section.
